(19) United States
(12) Patent Application Publication
(10) Pub. No.: US 2014/0086999 A1
(43) Pub. Date: Mar. 27, 2014

(54) BIOLOGICALLY ACTIVE DIETARY SUPPLEMENT FOR NORMALIZING THE ANDROGEN LEVEL IN MEN AND THE OVERALL CONDITION AND REDUCING OBESITY

(71) Applicant: Dmitry Gennadjevich Elistratov, Penza (RU)

(72) Inventor: Dmitry Gennadjevich Elistratov, Penza (RU)

(21) Appl. No.: 14/006,829

(22) PCT Filed: May 3, 2011
(86) PCT No.: PCT/RU11/00294

§ 371 Date: Nov. 18, 2013

(30) Foreign Application Priority Data
May 3, 2011 (RU) 2010117666

(51) Int. Cl. A61K 36/73; A61K 35/64

(52) U.S. Cl. CPC A61K 36/73; A61K 35/642
USPC 424/539

(57) ABSTRACT

Biologically active dietary supplements (BADS) are proposed for normalizing the hormone balance and the overall condition in men based on natural ingredients. The BADS contain white cinquefoil roots and rhizomes or the above-ground part of white cinquefoil, or a mixture thereof, as well as drone brood in a quantity of from 20 mass % to 80 mass %. The disclosure makes it possible to produce a product which demonstrates, by virtue of an optimally selected ratio of ingredients, a synergistic effect in relation to the normalization of the androgen level and the prophylaxis of obesity in men.

### CROSS-REFERENCE TO RELATED APPLICATIONS

This application is a U.S. national stage application of an international application PCT/RU2011/000294 filed on 3 May 2011, published as WO2012150872, which international application claims priority of a Russian Federation patent application RU2010117666 filed on 3 May 2011.

### FIELD OF THE INVENTION

The invention relates to food industry, namely, to biologically active dietary supplements (BADS) used for normalization of hormonal profile and general health in men and produced based on natural ingredients.

### BACKGROUND OF THE INVENTION

A prior art search on this matter showed the existence of large number of known herbal-based agents and promoting the normalization of hormonal profile and metabolic processes.

There is known a broad-spectrum treatment and prevention remedy containing propolis and milk thistle oil (Patent of Russian Federation No. 2175238, A 61 K 35/78). Due to the claimed combination of its ingredients, this biologically active supplement is effective in terms of its ergogenic action, stimulation of mental and physical activity and natural resistance of the body, and improvement of gastrointestinal, urinogenital and respiratory functions.

There is known a herbal-based dietary supplement used for prevention of pathological symptoms of climacteric syndrome (Patent of Russian Federation No. 2197975). This dietary supplement contains herbal constituents with hormone-like properties comprised by such groups of compounds as isoflavonoids, lignans and coumarins, and is intended for prevention of dyshormonal pathological symptoms of climacteric and post-climacteric syndrome in men and women. The drawback of this known dietary supplement is that it does not address the endocrine regulation in men, the disturbance of which largely determines the development of obesity in aging men, leading to a variety of health disorders.

There is known a biologically active product containing drone brood and used to maintain proper metabolism and nutrition of body tissues and organs in men (Drone Milk by Tentorium Apicompany—found on the Web: http://medovik.com/page_mol_{—}0.html, 25.01.2008).

The drawback of this known product is its low effect on metabolic processes in men and lack of data in regard to endocrine system regulation in men.

There is known a preventive remedy used for prevention of thyroid disorders and containing roots and rhizomes of white cinquefoil, or aerial portions of white cinquefoil, or their mixture (Patent of Russian Federation No. 2351334). The drawback of this known remedy is its low efficacy in normalizing the hormonal profile and general health in men.

### OBJECT AND SUMMARY OF THE INVENTION

It is the object of this invention to develop a dietary supplement to prevent obesity in men with partial age-related androgen deficiency, and to normalize their androgen levels.

The invention aims at expanding the range of biologically active dietary supplements provide a wide range of beneficial effects in the human body and promote rehabilitation of the population.

To achieve this object, potentiating the beneficial therapeutic effect of white cinquefoil was proposed by supplementing the formulation with drone brood as a donator of entomological sex hormones such as prolactin, estradiol, progesterone and testosterone stimulating the androgen functions in men. Rich in vitamins and hormones which are not hormone substitutes, drone brood is effective in treating hormonal disturbances. It offers a possibility of harmonizing the functioning of all organs and systems in the human body.

The objective is achieved by proposing a biologically active dietary supplement to normalize androgen levels in men, improve general health and reduce obesity, characterized in that it contains roots and rhizomes of white cinquefoil or aerial parts of white cinquefoil, or their mixture, and also contains drone brood in the following proportion: drone brood—20 wt % to 80 wt %.

A result of the claimed invention is the creation of a dietary supplement with an optimally selected proportion of ingredients, free of side effects and suitable to be most effectively used for normalization of metabolic processes and hormonal profile in men due to its bioactive properties.

The applicability of a combination of said ingredients is supported by the fact that combined use of agents of herbal and animal origin has been observed to potentiate the pharmacological activity of ingredients, because the effects exerted by constituents of white cinquefoil and drone brood occur by different pathways.

White cinquefoil normalizes metabolic processes in the body by normalizing the activity of the thyroid gland. Hypothyroid state causes a slowing down of the metabolic processes and, consequently, leads to obesity.

The effect of drone brood is provided by enzymes, amino acids, a complex of vitamins, macro- and micronutrients, hormones and growth factors stimulating the cell metabolism and boosting fat burning within the body.

This ensures the integrity and versatility of therapeutic effects in the human body and makes it possible to address both the disease (consequence) and its underlying cause.

Some causes of obesity in men are:
1. Androgen deficiency,
2. Hypothyroidism,
3. Disordered digestion.

Most often, the existing treatment methods involve hormone replacement therapy, which is the traditional allopathic approach to treatment. The risk of this method is determined by two factors: 1) hormone dosages should be carefully adjusted, 2) hormone replacement therapy suppresses the body's own production of hormones.

The claimed dietary supplement provides concurrent normalization of thyroid gland function (and, respectively, of hormones produced by it) and the level of androgens produced by testes, and regulates the digestive function through the action of chemical constituents of white cinquefoil.

### EXEMPLARY PREFERRED EMBODIMENTS OF THE INVENTION

Examples of the claimed BADS formulation:

### EXAMPLE 1

Cinquefoil roots—20 wt %

Drone brood—80 wt %

### EXAMPLE 2

Cinquefoil leaves—50 wt %

Drone brood—50 wt %

### EXAMPLE 3

Cinquefoil leaves—80 wt %

Drone brood—20 wt %

During the implementation of the objective, we revealed that the resulting action of the supplement was significantly more potent than that of each of its ingredients taken alone, due to a synergistic effect.

A six-month trial of the dietary supplement on 20 volunteers showed a significant decrease in the weight of fat deposits, reduction of dyspnea, normalization of heart rate, increase in general sense of well-being, improved sleep, enhanced sexual performance and normalization of hormonal profile.

The action of the claimed dietary supplement was compared to that of its individual ingredients.

Thus, 20 male volunteers, within the period of several months, received the claimed dietary supplement formulated as per Example 1 (1^{st} group); another 20 subjects received the dietary supplement formulated as per Example 3 (2^{nd} group); the 3^{rd} group of 20 subjects received cinquefoil in the form of powdered leaves, and 4^{th} group used the preventive remedy based on drone brood (Drone Milk) Hormonal androgen levels, body weight and general health condition were monitored in each group.

The study showed an increase in androgen levels in 90% of the subjects of the 1^{st} group and in 85% of the subjects of the 2^{nd} group, while in the 3rd and 4^{th} groups the increase in androgen levels over the same period of time was 40%, respectively; a decrease in fat deposits was observed in 70% of subjects of the 1^{st} group, 60% of subjects in the 2^{nd} group, 30% of subjects in the 4^{th} group; only 20% of volunteers in the 3^{rd} group experienced a decrease in body weight. Subjects of the 1^{st} and 2^{nd} groups reported a significant improvement in their general sense of well-being.

Therefore, the claimed supplement shows greater efficacy in normalization of metabolic processes and hormonal profile in men as compared to the separate use of each of its ingredients.

The efficacy of the dietary supplement is explained by the fact that concurrent regulation of two endocrine glands is more effective as compared to regulating either of these functions alone. A characteristic effect of thyroid hormones is their ability to stimulate the energy metabolism by enhancing the oxidizing processes in the mitochondria, increasing the oxygen consumption and carbon dioxide elimination and increasing the utilization of carbohydrates, fats and proteins, which leads to weight loss and intensive uptake of glucose from the bloodstream.

It should be noted that fat tissue is currently viewed by physicians as an endocrine organ, and its excess is believed to adversely affect not only the metabolic rate, but also the testosterone production in men. All obese men have a relative or absolute decrease in the secretion of testosterone, and treating obesity is impossible without normalizing this function. Therefore, the use of drone brood influences the androgenic function in men and resists the action of fat tissue. The thyroid does not affect the endocrine functions of fat tissue.

The claimed dietary supplement is produced in powder, pill or capsule form, and may also be produced in the form of aqueous alcoholic extract and dosage forms prepared based on such extract, namely: powder, pills and capsules.

Therefore, despite the fact that all ingredients in the composition of the claimed supplement are known in folk and traditional medicine, their combination in a single product is not known from the prior art; namely, the revealed synergistic effect made it possible to accomplish the objective and to achieve the claimed result of creating a dietary supplement with an optimally selected proportion of ingredients, free of side effects and effective in prevention of obesity in men and normalizing their androgen levels.

1. A biologically active dietary supplement for normalizing the androgen levels in men, improving general health and reducing obesity, said dietary supplement comprises:
(a)—roots and rhizomes of white cinquefoil, or
aerial portions of white cinquefoil, or
mixture thereof; and
(b) drone brood in the weight proportion of: from 20 wt % to 80 wt %.
